(19) United States
(12) Patent Application Publication
(10) Pub. No.: US 2004/0225312 A1
(43) Pub. Date: Nov. 11, 2004

(54) Linearly lancing integrated pivot disposable

(75) Inventors: Eugene Orloff, Berkeley, CA (US); Kumar Subramanian, Pleasanton, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Phoenix Bioscience, Pleasanton, CA

(21) Appl. No.: 10/435,409

(22) Filed: May 9, 2003

(51) Int. Cl. A61B 17/14

(52) U.S. Cl. 606/182

(57) ABSTRACT

A sample retrieval device for collecting fluid from skin. The sample retrieval device has a lancing device and an analytic device integrated into a single body, the analytic device residing in a sample retrieval portion of the body. The body is also equipped with a signal pathway and an engagement portion. The lancing device and the fluid inlet in the sample retrieval portion are positioned in the body so that the fluid released by the lancing device is taken up into the fluid inlet by having the body direct the lancing device and the sample retrieval portion to the fluid released from the skin by pivoting.

### CROSS-REFERENCES TO RELATED APPLICATIONS

NOT APPLICABLE

### STATEMENT AS TO RIGHTS TO INVENTIONS MADE UNDER FEDERALLY SPONSORED RESEARCH OR DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND OF THE INVENTION

1. Field of the Invention

The present invention relates to a device for obtaining body fluid samples, and more particularly, to a disposable sample retrieval device that linearly lances a subject and gathers the sample by a pivoting motion.

2. Description of the Prior Art

The art of body fluid sampling is inundated with a wide variety of methods and instruments. By traditional techniques, in order to perform an analysis on biological fluid, a patient is required to make a trip to their physician's office and donate a body fluid sample. This is usually in the form of blood or urine that is subsequently delivered to a laboratory and eventually analyzed. In order to obtain a blood sample, it is most common for a hypodermic to be inserted into a vein to gather a large volume of blood, the majority of which often goes unused. More recently, blood tests and analyzers have been developed which require the pricking of a finger to supply a drop of blood, which is analyzed by a separate unit.

In recent years there has been a growing need to provide the ability to perform body fluid analysis “on the spot”, without having to send an individual to a physicians office and wait for laboratory results. Law enforcement and military personnel have pressing needs to be able to perform on the spot testing for drugs of abuse and exposure to CBR (chemical biological and radiological) warfare. Furthermore, in order to enhance the quality of life and for proper management of health, there has been a push to equip the common individual to perform biological fluid testing at home or during normal day-to-day routines, without having to visit their physician. Several types of instruments have been developed along the lines of home pregnancy testers, hemoglobin testers, and blood glucose testers for diabetics.

Diabetes mellitus is a chronic disease that affects more than 15 million Americans. About seventy five percent of these are type 2 (non-insulin dependent). Accurate blood glucose monitoring is required for proper management of blood sugar levels for diabetics. Several systems have been developed over the recent years permitting home testing of blood sugar levels. Most of these systems require the user to draw a blood sample usually from the fingertip and deliver the blood sample to a collection device in the form of a capillary and reservoir with predisposed reagents for analysis. Due the sensitivity of the fingertips however, testing is quite painful and even traumatic for many users, especially among children and infants. Recently devices have been developed which sample body fluid from the forearm as a means of drawing body fluid painlessly, U.S. Pat. Nos. D0427312, US06,120,676, USD0426,638, USD0424,696. However, obtaining the volume of blood required for these systems from the forearm has been difficult.

The industry of point of care blood glucose monitors offers many varieties of instruments and sampling devices, available in a large range of designs. Most blood glucose analyzers consist of two separate major components: a skin penetrating instrument, known as the lancing device, and a blood collection/analysis instrument, known as the meter. Accordingly, these blood glucose instruments require two distinctly different steps: a lancing step and a filling step, the filling requiring manual delivery of a relatively large volume of body fluid to the collection device. The proper delivery of the blood sample to the collection device often requires a good deal of dexterity and is quite difficult for older diabetics, and individuals with failing eyesight. Often the blood drop ends up smeared along the collection device or on the user, creating a mess as well as a failed test. As a result tests often need to be repeated several times until the procedure is performed properly. This is a prevalent shortcoming of the current art.

Integration of the skin-penetrating device with the fluid collecting/analyzing device of body fluid analyzers is uncommon in the industry. A device that integrates the skin penetrating and fluid collecting instruments and procedures has many advantages. Integrated systems do not require as large as a volume of body fluid to be produced as required for non-integrated systems. Integrating the skin-penetrating device with the fluid collection/analyzing device creates an integrated device suited towards automatic delivery of the body fluid sample. Integrated and automated systems may demonstrate a high level of precision in collecting the sample, allowing for smaller sample sizes to be generated. Smaller sample sizes result in smaller lancet sizes, less pain and trauma for the user, and fewer if any failed tests. Furthermore, an integrated skin-penetrating and fluid collecting/analyzing device consists of only one major component, reducing the number of components and steps a user has to accommodate.

Several companies and individuals have developed various devices in an attempt to integrate the sample production and collection devices and automate the process. However, there are few, if any, truly integrated skin penetrating and collecting devices currently available.

One such device described by Smart and Subramanian, U.S. Pat. No. 5,801,057, refers to a silicon microsampler. The silicon microsampler is a microchamber forming a cuvette with an integrated hollow silicon needle. The microchamber and needle are formed from one silicon substrate through a series of etching processes. The microchamber and microneedle of the microsampler are covered with a glass layer that is anodically bonded to the silicon portion. The microsampler is filled by inserting the microneedle into the skin; under the skin surface, the microneedle contacts a blood sample and draws it into the microcuvette for analysis.

U.S. Pat. No. 4,873,993, Cuvette, assigned to Personal Diagnostics Inc., describes “a cuvette with or without a lancet secured thereto and extending therefrom for producing skin puncture to produce body fluid of interest, the cuvette is made of optically transparent material and is provided with a shape and a plurality of optical elements such as integrally formed optical elements for causing a light beam to pass therethrough by total internal reflectance and for causing the beam of light to be reflected back along a line different from the direction of the line of entry of the beam of light into the cuvette such as back along a line generally parallel to the line of entry of the beam of light into the cuvette and in the opposite direction to the direction of entry of the beam of light into the cuvette.” This device is used with an instrument that performs the lancing operation, U.S. Pat. No. 5,029,583. However, the lance and cuvette are not attached in such a way to facilitate automated filling of the cuvette. The cuvette filled manually by the patient “wiping” the body fluid sample across the opening to the cuvette.

Another device described by Garcia et al., U.S. Pat. No. 4,637,403, refers to a hand-held portable medical diagnostic device. The system utilizes a “disposable needle or lance probe package which carries a chemical reagent strip.” The disposable is used within an instrument utilizing “a spring arrangement for actuating a needle or lance into the skin for transferring blood from a finger or other area to the chemical reagent strip.” The instrument and actuation system may also create a vacuum at the lance site to help move blood from the wound to the test strip.

Another application described by Douglas et al., U.S. Pat. No. 6,099,484, refers to methods and apparatus for sampling and analyzing body fluid. The device described is an instrument containing a lancet for making an incision, a capillary tube for drawing up fluid, and a test strip affixed to the capillary tube. Various embodiments of the instrument and invention are described. The instrument contains all components mentioned and contains an actuating system that lances and places the capillary at the lance site. Another embodiment describes the instrument first placing a test strip at the skin surface and then piercing the skin through the test strip with a lancet.

Another device by Douglas et al. U.S. Pat. No. 6,183,489 describes a macro collection device where the fluids are drawn up by a capillary tube to an external analytic test strip.

Another device described by Frederick L. Dechow, U.S. Pat. No. 4,883,068, refers to a blood sampling device and method. The instrument consists of a double-sided cannula and a reservoir with a penetrable end cap axially aligned in a compressible device. The device is laid upon the skin and compressed. This motion causes the cannula to first puncture the skin surface and then the reservoir end cap. In this position blood is then delivered to the reservoir.

### SUMMARY OF THE INVENTION

The present invention provides a Linear Lancing Integrated Pivot Disposable (LLIPD), generally referred to as a sample retrieval device, for collecting fluid from skin. Broadly, the sample retrieval device has a lancing device and an analytic device integrated into a single body, the analytic device residing in a sample retrieval portion of the body. The body is also equipped with a signal pathway and an engagement portion. The lancing device and the fluid inlet in the sample retrieval portion are positioned in the body so that the fluid released by the lancing device is taken up into the fluid inlet by having the body direct the lancing device and the sample retrieval portion to the fluid released from the skin. The body is preferably plastic and is fabricated using plastic injection molding.

More specifically the invention comprises an integrated lancing and analytic device for collecting fluid from skin, said device having a lancing device and an analytic device integrated into a single body wherein: i. the lancing device has a tapered skin penetrating portion extendible from the body so that it penetrates the skin to release fluid when the body of the device is moved linearly towards the skin; ii. the analytic device is housed in a sample retrieval portion of the body, the analytic device comprising an inlet, an analytic region having capillary dimensions, and an outlet wherein the analytic region is positioned in fluid communication between the inlet and outlet and a signal pathway in the body transmits a signal of analysis from the analytic region to the outside, optionally the signal is not visible to a user; iii. wherein the lancing device and the sample retrieval portion are positioned in the body about a pivot point at an angle of less than 180 degrees, and iv. wherein penetrating the lancing device into a subject by moving the lancing device along a linear axis defined by the lancing device, retracting the lancing device from the subject along the linear axis to release fluid from the skin, and pivoting the sample retrieval device so that the sample retrieval portion of the body is adjacent the location on the subject penetrated by the lancing device, causes the fluid released by the skin to enter the analytic device.

The lancing device is an object used to penetrate the skin, less than 3 mm in depth, preferably having the form of a needle. The lancing device is tapered at one end, coming to a point with sufficient sharpness for penetrating skin. The lancing device is preferably fabricated from metal but may also be fabricated from plastic. In a preferred embodiment the lancing device is a metal needle, protruding cylindrically from the body with one end tapered to a point. In an alternative embodiment the lancing device may be metal having the shape of a razor edge.

The analytic device is used to pull body fluid off from the skin surface and perform an analysis on the fluid. The fluid may be blood, interstitial fluid, or a combination of both, having a volume range of 50-300 nanoliters. The analytic device comprises an inlet, an analytic region, and an outlet wherein the analytic region is positioned in fluid communication between the inlet and outlet. A signal pathway in the body of the sample retrieval device transmits a signal of analysis from the analytic region of the analytic device to the outside. The signal pathway may be either a pair of electrodes or an optical signal. The analytic device is located in the sample retrieval portion of the body. In one embodiment the body may create a fluid seal with the analytic device by using tabs to provide mechanical pressure or an interference fit. The analytic device may be fabricated from either silicon, plastic or glass, or any combination of the three.

One object of the present invention is to provide an instrument system comprising an instrument housing containing an integrated lancing and analytic device for collecting fluid from skin. The instrument housing is fitted to removably house the device having a lancing device and an analytic device integrated into a single body. The lancing device has a tapered skin-penetrating portion which extends from the body so that it penetrates the skin and retracts to release fluid from the skin when the instrument housing is held to the skin; the analytic device comprises an inlet, an analytic region, and an outlet wherein the analytic region is positioned in fluid communication between the inlet and outlet and a signal pathway in the body transmits a signal of analysis from the analytic region to the outside; and the lancing device and inlet of the analytic device are positioned in the body so that the fluid released by the lancing device is taken up into the inlet without removing the instrument housing from the skin. Through an engagement portion, the sample retrieval device may be coupled to a transport instrument and an analytic instrument located within the instrument housing. Furthermore the sample retrieval device may be spring loaded within the instrument housing, as well as driven by an electromechanical device within the instrument housing.

Another object of the present invention is to provide a method of collecting and analyzing fluids from skin. The method comprises four steps: 101 positioning on the skin of an animal an instrument housing containing a removable, integrated lancing and analytic device for collecting fluid from skin said device having a lancing device and an analytic device integrated into a single body; 102 linearly inserting and retracting the lancing device into and out from the skin to release fluid; 103 pivoting the sample retrieval device to position the sample retrieval portion of the body adjacent to the location on the skin penetrated by the lancing device; 104 analyzing the fluid that enters the analytic device through the fluid inlet of the sample retrieval portion.

The fluid in the analytic device may be analyzed using several methods: coulometric, optical, electrochemical, fluorescence, or chemiluminescence. The preferred analyte for the fluid is glucose.

Unless otherwise stated, all physical variations of the devices described herein are applicable to the methods of uses described herein.

### BRIEF DESCRIPTION OF THE DRAWINGS

FIG. 1 is a side view of a sample retrieval device in accordance with the present invention, wherein a analytic device of the sample retrieval device consists of two members.

FIG. 2 is a perspective view of the sample retrieval device illustrated in FIG. 1.

FIG. 3 is a perspective view of the sample retrieval device illustrated in FIG. 1, wherein the analytic device consists of two members and is illustrated retracted from the device, and the lancet is shown with a twist-off cap sized for automatic removal.

FIG. 4 is a perspective view of the sample retrieval device illustrated in FIG. 1, and the lancet is shown with a twist-off cap sized for manual removal.

FIG. 5 is a side view of a sample retrieval device in accordance with the present invention, wherein both the analytic device and a body of the sample retrieval device consist of two members, and the two members of the body have not yet been joined.

FIG. 6 is a perspective view of the sample retrieval device illustrated in FIG. 5.

FIG. 7 is a perspective view of the sample retrieval device illustrated in FIG. 5, wherein the analytic device is shown retracted from the body and the two members of the body have not yet been joined.

FIG. 8 is a perspective view of the sample retrieval device illustrated in FIG. 5, wherein the analytic device is shown retracted from the body and the two members of the body have been joined.

FIG. 9 is a perspective view of a sample retrieval device in accordance with the present invention, wherein the analytic device consists of one member and the body consists of two members, and wherein the analytic device is shown retracted from the body and the two members of the body have not yet been joined.

FIG. 10 is a perspective view of the sample retrieval device illustrated in FIG. 9, wherein the analytic device is shown retracted from the body and the two members of the body have been joined.

FIG. 11 is a perspective view of the sample retrieval device illustrated in FIG. 9, wherein the two members of the body have been joined.

FIG. 12 is a perspective view of a sample retrieval device in accordance with the present invention, wherein the analytic device consists of two members shown in exploded form retracted from the body.

FIG. 13 is a perspective view of the sample retrieval device illustrated in FIG. 12.

FIG. 14 is a perspective view of the sample retrieval device, wherein the body is formed from two members, wherein the two members have not yet been joined, and wherein the analytic device is formed from the two members of the body directly.

FIG. 15 is a perspective view of the sample retrieval device illustrated in FIG. 14.

FIG. 16 is a perspective view of a sample retrieval device in accordance with the present invention, wherein the analytic device is formed from one member, the body is formed from two members, the two members of the body have not yet been joined, and one member of the body reveals the sensor and electrical signal pathway.

FIG. 17 is a perspective view of a sample retrieval device in accordance with the present invention, wherein the analytic device is formed from one member, the body is formed from two members, the two members of the body have been joined, and the contact pads are revealed on the periphery of the body.

FIG. 18 is a top view of one member of the body of a sample retrieval device in accordance with the present invention, wherein the sensor, electrical signal pathway and contact pads are displayed.

FIG. 19 is a side view of a sample retrieval device in accordance with the present invention, wherein the analytic device consists of one member and the optical signal pathways are displayed.

FIG. 20 is a side view of a sample retrieval device in accordance with the present invention, wherein the analytic device consists of two members.

FIG. 21 is a perspective view of the sample retrieval device in accordance with the present invention and a transport instrument.

FIG. 22 is a perspective view of the sample retrieval device and the transport instrument wherein the body of the sample retrieval device is coupled to the transport instrument.

FIG. 22A is a perspective view of an alternate embodiment of the sample retrieval device wherein the analytic device consists of two members and the lancet is shown with a twist-off cap sized for automatic removal.

FIG. 22B is a perspective view of the sample retrieval device illustrated in FIG. 22A, wherein the analytic device is exploded from the body of the sample retrieval device.

FIG. 22C is a perspective view of the sample retrieval device illustrated in FIG. 22A, wherein the analytic device consists of 1 member, and the analytic device is shown exploded from the body of the sample retrieval device.

FIG. 23 is a top view of one member of an analytic device for use with a sample retrieval device in accordance with the present invention.

FIG. 24 is a side view of a analytic device for use with a sample retrieval device in accordance with the present invention, wherein the analytic device consists of two members.

FIG. 25 is a side view illustrating a sample retrieval device in accordance with the present invention, wherein the analytic device is adjacent to an analytic instrument and the body of the sample retrieval device is positioned inside an instrument housing with the linear axis of the lancing device normal to the skin surface.

FIG. 26 is a side view of the arrangement illustrated in FIG. 25, wherein the lancing device is inserted into the skin surface.

FIG. 27 is a side view of the arrangement illustrated in FIG. 25, wherein the lancing device is retracted from the skin surface and body fluid has been expelled from the skin surface.

FIG. 28 is a side view of the arrangement illustrated in FIG. 25, wherein the body has been pivoted and the sample retrieval portion and the analytic device are adjacent to the body fluid at the location penetrated by the lancing device.

### DETAILED DESCRIPTION OF THE INVENTION

The present invention provides a sample retrieval device 100 that may be referred to as a Linearly Lancing Integrated Pivot Disposable (LLIPD). In summary, the device of the present invention combines both a lancing device 102 and an analytic device 103 in such a way that they may be used together conveniently for lancing a skin surface and drawing a small sample of body fluid. In all embodiments of the invention the lancing device 102 and analytic device 103 are combined together, integrated in a body 101.

The body is the housing for lancing device 102 and analytic device 103. The body holds and locates both the lancing device and the analytic device in proper orientation with respect to each other. A preferred material for the body is plastic. The body is preferably fabricated by plastic injection molding.

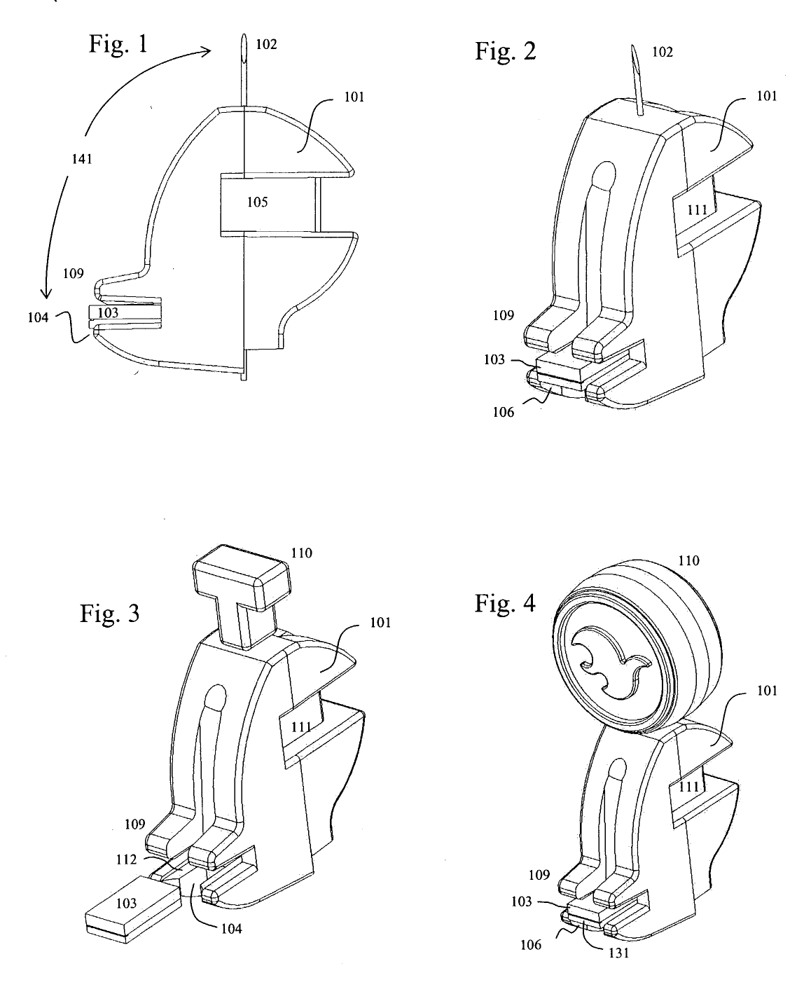

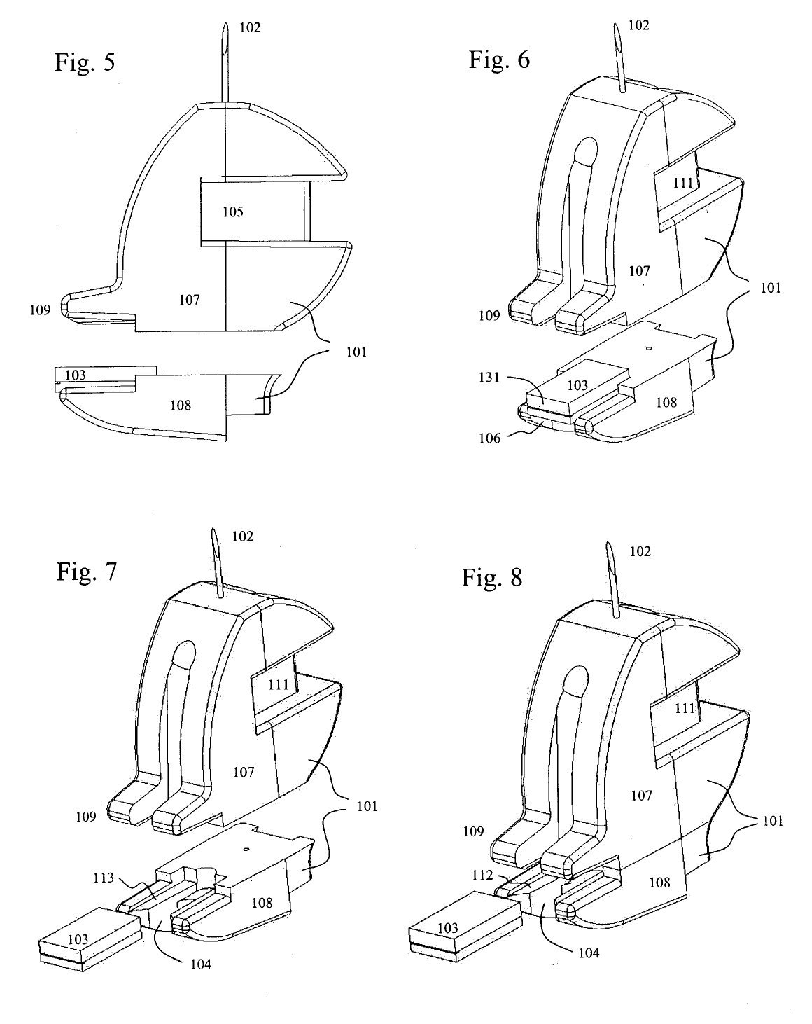

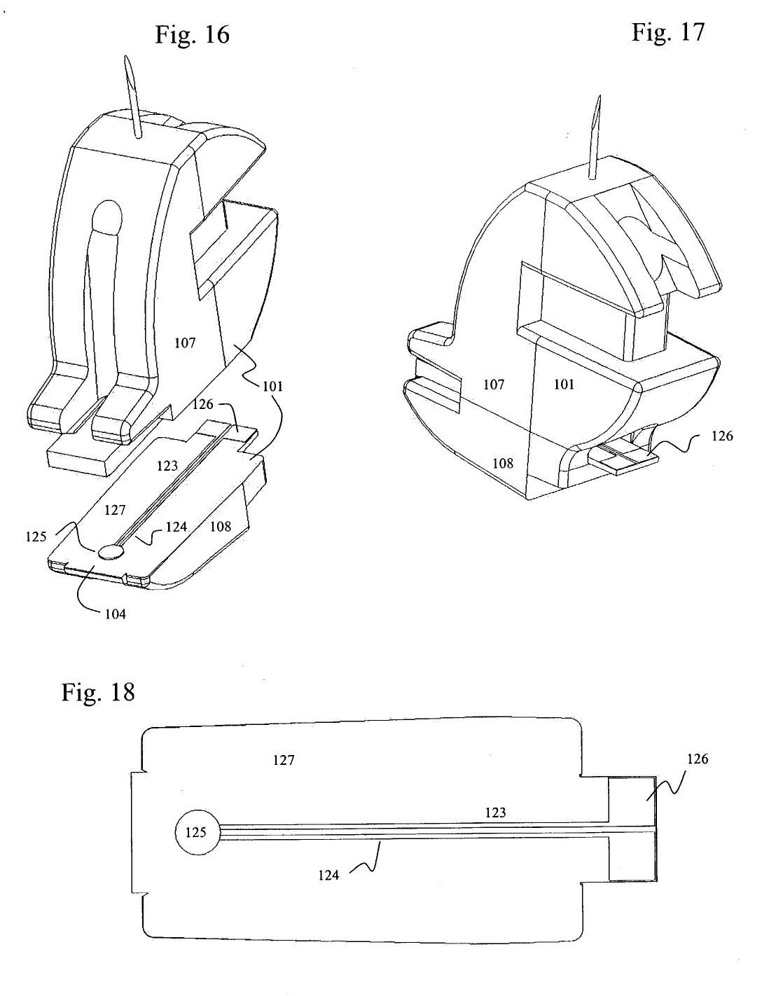

As illustrated in FIGS. 1-4, body 101 may be formed from one single piece. Similarly, as seen in FIGS. 5-8, the body may be formed from two pieces 107, 108 that are joined during its assembly. A preferred method for forming the body is from the two separate pieces as shown in FIGS. 5-8. The general shape of the body is preferably to be elliptical or circular in nature having a diameter ranging from 5 mm to 15 mm and a thickness ranging from 3 mm to 6 mm. A preferred diameter for the body is 7.5 mm. A preferred thickness for the body is 3.5 mm. When the body is formed from two pieces 107, 108 they may be joined by sonic welding, gluing, press fitting, or other means known to those skilled in the art. The body incorporates several features and regions. Body 101 has a sample retrieval portion 104, tabs 109, a fluid inlet portion 106, a twist-off cap 110, a pivot point 105, an engagement portion 111, and a signal pathway shown in FIGS. 16-19.

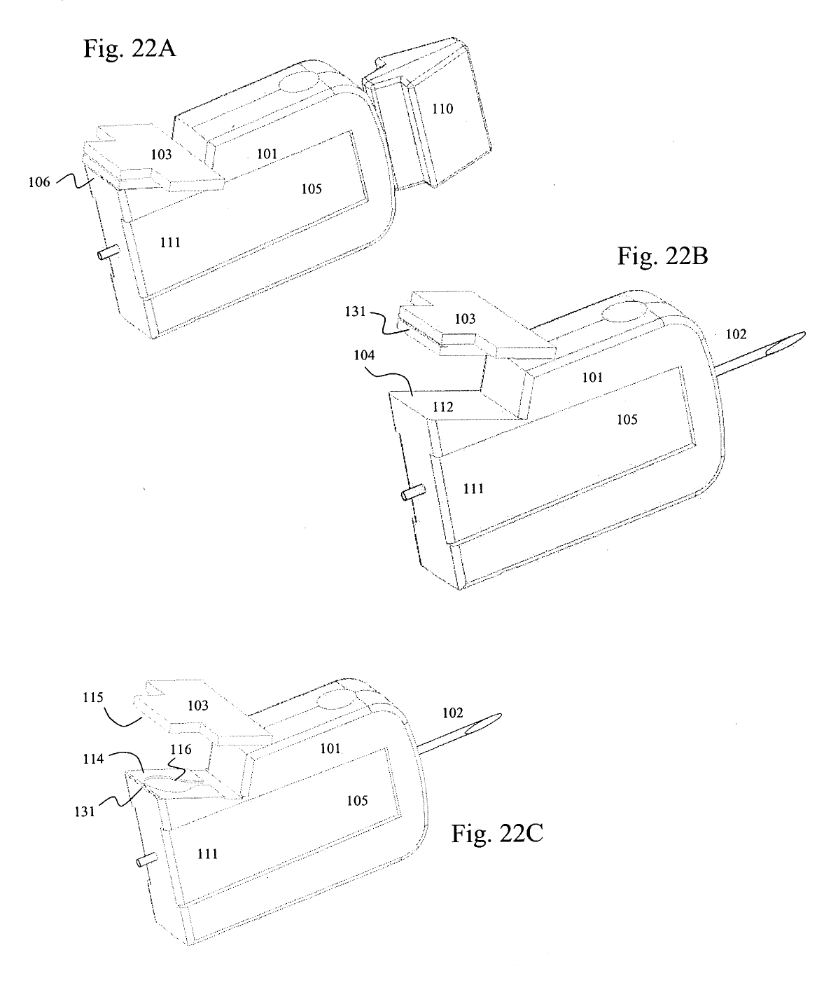

FIGS. 22A-22C illustrate an alternate preferred embodiment of body 101. In this embodiment body 101 is rectilinear in shape and is formed from 1 single piece. In this embodiment body 101 may have a thickness ranging from 1.5 mm to 5 mm, a length ranging from 5 mm to 10 mm, and a width ranging from 3 mm to 7 mm. In this embodiment the preferred dimensions for body 101 are 2 mm thick, 7 mm long, and 4.5 mm wide. As illustrated in FIGS. 22A-22C, body 101 has a sample retrieval portion 104, a fluid inlet portion 106, a twist-off cap 110, a pivot point 105, an engagement portion 111, and a signal pathway.

Referring to FIGS. 1-8, the body may have a sample retrieval portion 104 formed during the injection molding process. The sample retrieval portion is the portion of the body that receives the fluid sample and houses an analytical device 103. As seen in FIGS. 3-4, the sample retrieval portion may be defined by a slot 112 and may be equipped with tabs 109, both the slot 112 and the tabs 109 being sized to hold the analytic device in an interference fit. When the body is formed from one piece, as seen in FIGS. 1-4, the slot and tabs may be formed during the injection molding process. In this embodiment the slot may have a width ranging from 1 mm to 5 mm, a height ranging from 0.350 mm to 2 mm, and a depth ranging from 1 mm to 10 mm. The preferred dimensions for the slot are 2 mm in width, 1 mm in height, and 2 mm in depth. The tabs may have a size ranging from 1 mm to 6 mm wide, 1 mm to 3 mm thick, and 1 mm to 3 mm long. The tabs are preferably 3.5 mm wide, 1.5 mm thick, and 2 mm long.

In a preferred embodiment of the invention, body 101 is formed from two separate pieces 107, 108, as seen in FIGS. 5-8. In this embodiment one of the pieces 108 may have a depression 113, illustrated in FIG. 7, formed during the molding process that creates slot 112, defining sample retrieval portion 104, when the two pieces of the body are joined together. In this embodiment depression 113 may have a width ranging from 1 mm to 5 mm, a height ranging from 0.350 mm to 2 mm, and a depth ranging from 1 mm to 10 mm. The preferred dimensions for the depression are 2 mm in width, 1 mm in height, and 2 mm in depth. In all embodiments of the present invention the sample retrieval portion is positioned within the body such that an angle 141 between lancing device 102 and sample retrieval portion 104 may range from 0° to 180° about a pivot point 105 within the body, as shown in FIG. 1. The preferred angle 141 between the sample retrieval portion and the lancing device is 135°.

In the embodiments of the invention as illustrated in FIGS. 1-8 the entrance to slot 112 is defined as fluid inlet portion 106. The fluid inlet portion is the area through which fluid is introduced into the sample retrieval portion, and is defined by the body. When of the form illustrated in FIGS. 1-8, the fluid inlet portion has cross sectional dimensions identical to those of the slot defining the sample retrieval portion. An alternative embodiment of the fluid inlet portion is illustrated in FIGS. 14-15. In this embodiment fluid inlet portion 106 is integral with a capillary channel 116 formed into one surface 122 of one member 108 of the body. In this embodiment the fluid inlet portion has dimensions suitable for causing fluid to flow within itself due to capillary forces. In this embodiment the fluid inlet portion may have a width ranging from 1 mm to 6 mm and a height ranging from 25 μm to 150 μm. In this embodiment the preferred dimensions for fluid inlet portion 106 are 3.5 mm wide and 50 μm in height.

As seen in FIGS. 3-4, the body may incorporate a protective twist-off cap 110 covering the lancing device. The twist-off cap may be formed as an integral part of the body during the plastic injection molding process. The twist-off cap may be sized to allow for either manual or automatic removal in order to expose the lancing device. The twist-off cap is preferably sized for automatic removal as illustrated in FIG. 3. When the twist-off cap is sized for automatic removal it may have the general shape of a “T”. In this embodiment the twist-off cap may have a width ranging from 1 mm to 3 mm at the lower end, a width ranging from 3 mm to 6 mm at the upper end, a thickness ranging from 1 mm to 3 mm and a height ranging from 2 mm to 5 mm. When sized for automatic removal the twist-off cap is preferably 1.5 mm wide at the lower end, 3.5 mm wide at the upper end, 2 mm thick, and 3.5 mm in height. When the twist-off cap is sized for manual removal as shown in FIG. 4, the twist-off cap may have a thickness ranging from 3 mm to 5 mm, and a diameter ranging from 5 mm to 10 mm. The preferred dimensions for a manually removable twist-off cap are 3.5 mm thick, and 8 mm in diameter.

FIG. 22A, shows an alternate version of twist-off cap 100 for an alternate preferred embodiment of the sample retrieval device. In this embodiment the twist-off cap is sized and shaped for automatic removal. In this embodiment twist-off cap 110 may have a width ranging from 0.65 mm to 2 mm at a lower end, a width ranging from 1 mm to 3 mm at an intermediate location, a width tapering a point at an upper end, a thickness ranging from 1 mm to 4 mm and a height ranging from 2 mm to 4 mm. In this embodiment preferred widths are 0.65 mm, 1 mm, and tapering to a point at a lower end, intermediate end, and an upper end respectively. The preferred thickness is 3 mm. The preferred height is 2.75 mm.

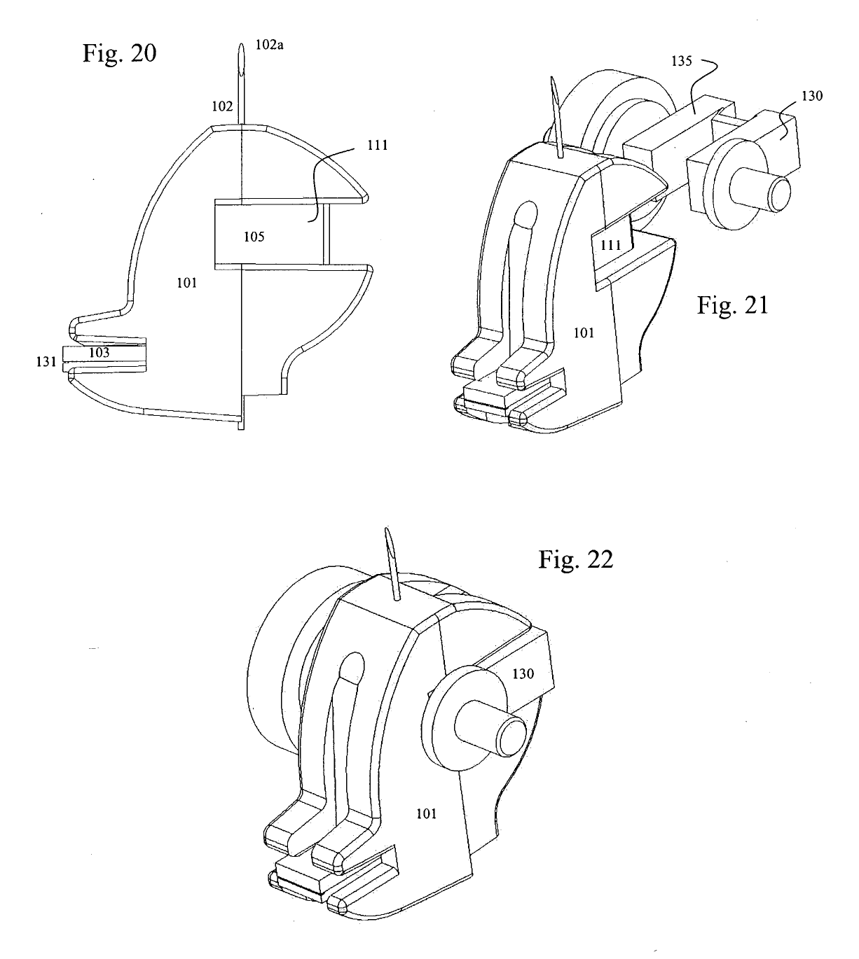

The body of the sample retrieval device may also incorporate an engagement portion 111, as illustrated in FIGS. 20-22. The engagement portion is defined by the surface of the body that couples directly to a transport instrument 130. The body, through the engagement portion is attached to and removed from the transport instrument before and after a test. The engagement portion may be in the form of a slot, a hole, tabs or other common attachment means that are designed dimensionally to mate to a receptacle in the transport instrument. The engagement portion is formed into the body during the plastic injection molding process. As shown in FIGS. 20-22 when the engagement portion is in the form of a slot the engagement portion may have a width ranging from 1 mm to 3 mm, a height ranging from 1 mm to 3 mm, and a length ranging from 2 mm to 6 mm. The preferred dimensions for the engagement portion when in the form of a slot are 2 mm wide, 1.5 mm in height, and 5 mm long. When the engagement portion is in the form of a hole the engagement portion may have a diameter ranging from 1 mm to 3 mm, preferably 2 mm.

Engagement portion 111 may also act as a pivot point 105 in body 101. The pivot point is a point within the body about which the body may rotate angularly. When pivoted about the pivot point the body may rotate in a range from 0°-360°. The preferred angular rotation about the pivot point is 225°.

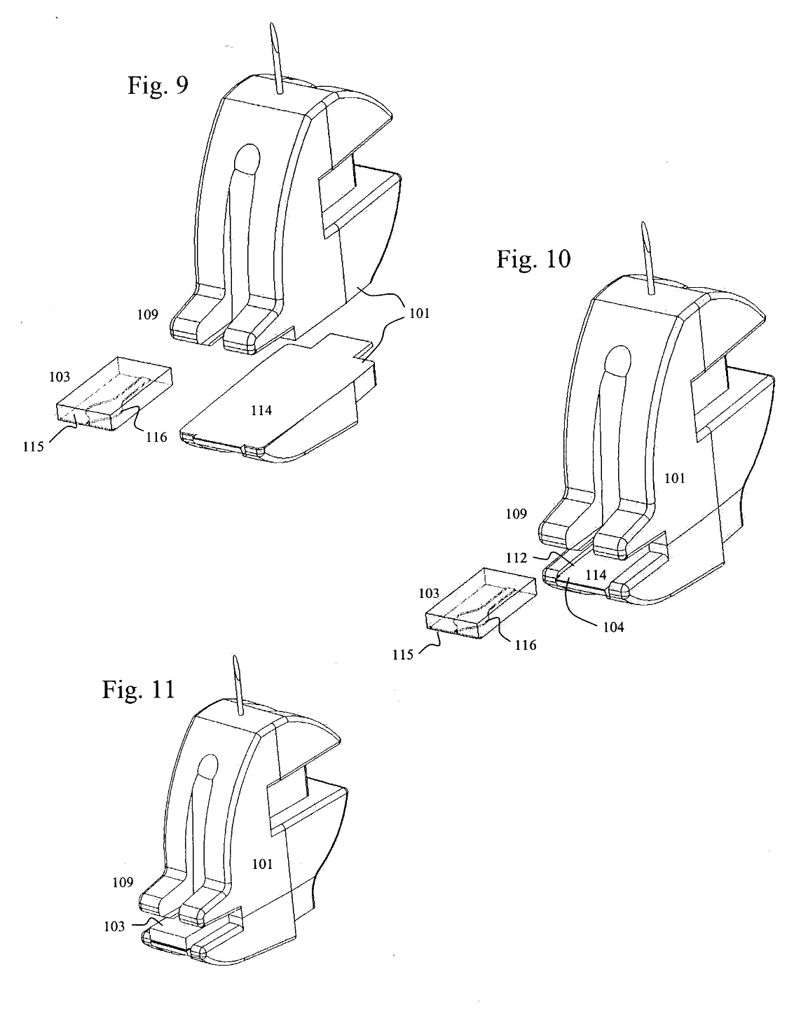

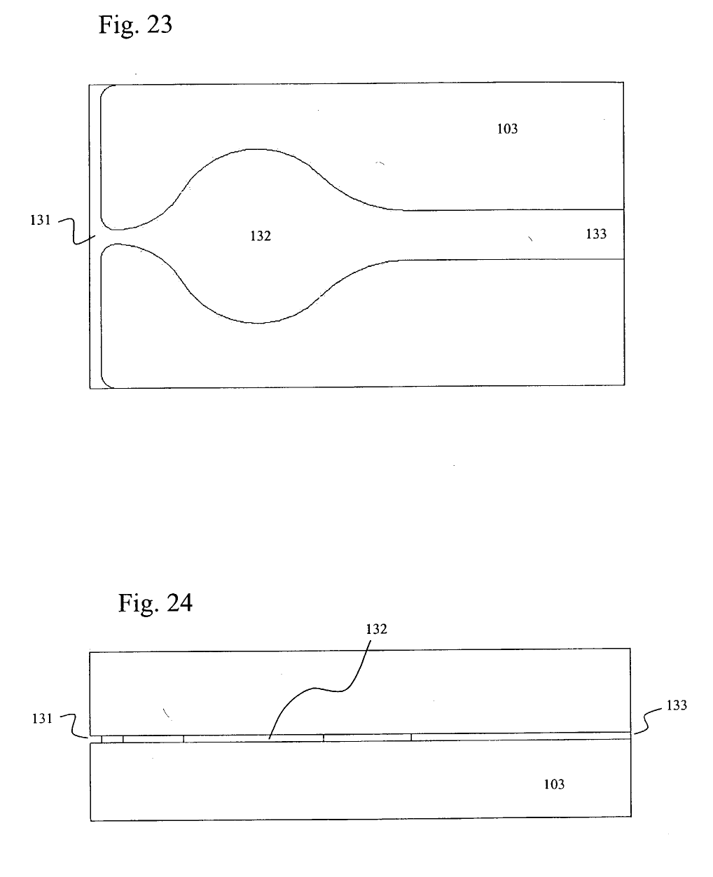

The sample retrieval device contains an analytic device 103. The analytic device is a device for collecting and analyzing body fluid. As illustrated in FIGS. 23-24, the analytic device comprises an inlet 131 an analytic portion 132 and a vent 133, the analytic portion being in fluid communication with the inlet and vent. The inlet, the analytic portion and the vent make up a capillary channel in the analytic device. The inlet to the analytic device may range from 1 mm to 6 mm wide. The analytic portion may range from 0.500 mm to 2 mm wide. The vent may range from 100 μm to 500 μm wide. The preferred dimensions for the inlet, analytic portion and vent of the. analytic device are 2 mm, 1 mm and 325 μm respectively. The depth of the capillary channel may range from 20 μm to 150 μm. The preferred depth for the capillary channel is 50 μm. Analytic device 103 may be fabricated from silicon, plastic, glass or any combination of the three. The analytic device may exist in a variety of embodiments. As shown in FIGS. 9-13, the analytic device may be formed from either one or two members 119, 120 and inserted into sample retrieval portion 104 of body 101. When the analytic device is formed from one or two separate members and inserted into the body, the two members may have dimensions ranging from 1 mm to 5 mm wide, 1 mm to 5 mm long, and 0.350 mm to 0.750 mm thick. The volume for the analytic device may range from 50 nl to 300 nl. The preferred dimensions for the members of the analytic device are 2 mm wide, 2.5 mm long and 0.500 mm thick, with a preferred volume of 60 nl. Alternatively, the analytic device may be formed from one member 108 of the body as shown in FIGS. 14-15. When the analytic device is formed from one member 108 of the body as shown in FIGS. 14-15, inlet 131, analytic portion 132 and vent 133 have the same dimensions and volumes as described above.

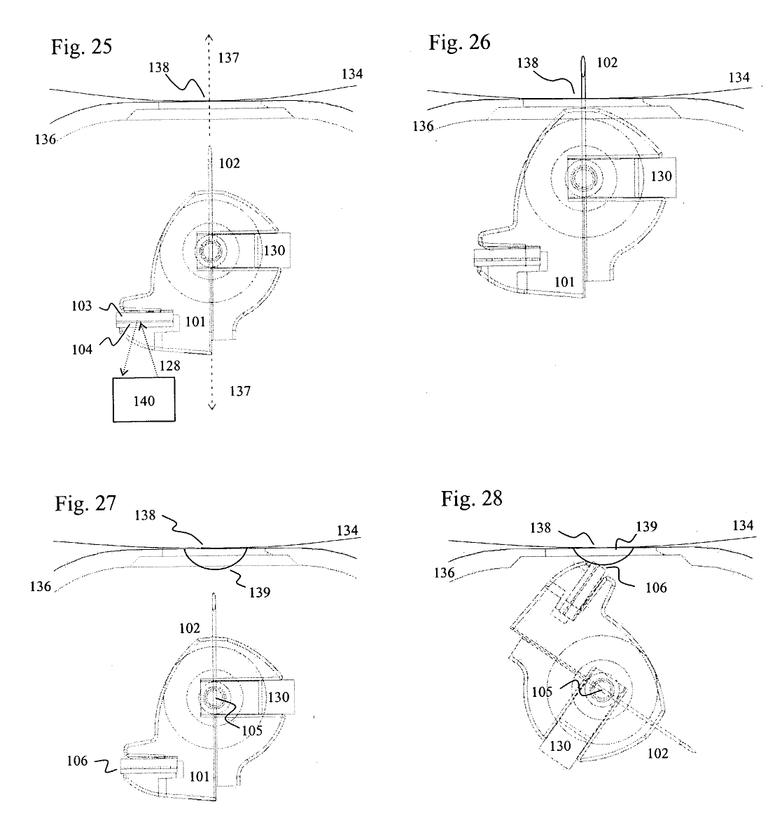

As illustrated in FIGS. 25-28, body 101 of the sample retrieval device contains a lancing device 102. The lancing device is a device for penetrating skin 134 to a depth sufficient to induce body fluid 139 to well up to the skin surface 134 when the body is moved linearly to the surface of the skin. The lancing device is tapered at one end, coming to a point with sufficient sharpness for penetrating skin 134. The lancing device protrudes from the body 101 0.5 mm-3.5 mm. The preferred protrusion distance is 2.75 mm. The lancing device may have a diameter ranging from 100 μm to 750 μm. The preferred diameter is 320 μm. The lancing device is preferably fabricated from metal but may also be fabricated from plastic. In a preferred embodiment the lancing device is a metal needle, protruding cylindrically from the body with one end tapered to a point. In an alternative embodiment the lancing device may be metal having the shape of a razor edge.

Referring to FIG. 20, in all embodiments of the invention the linear distance from pivot point 105 to fluid inlet 131 of analytic device 103 exceeds the linear distance from pivot point 105 to tip 102a of lancet 102. The amount by which the linear distance from pivot point 105 to fluid inlet 131 of analytic device 103 exceeds the linear distance from pivot point 105 to tip 102a of lancet 102 may have a range of 0.5 mm to 2.0 mm. The preferred amount by which the linear distance from pivot point 105 to fluid inlet 131 of analytic device 103 exceeds the linear distance from pivot point 105 to tip 102a of lancet 102 is 1.0 mm.

### EXAMPLES

The following examples are provided by way of illustration only and not by way of limitation. Those of skill will readily recognize a variety of noncritical parameters that could be changed to yield essentially similar results.

The device of the present invention is a Linearly Lancing Integrated Pivot Disposable (LLIPD) and is generally referred to generally as a sample retrieval device 100. In summary, the device of the present invention combines both a lancing device 102 and an analytic device 103 in such a way that they may be used together conveniently for lancing a skin surface and drawing a small sample of body fluid. In all present embodiments of the present invention the lancing device and analytic device are combined together, integrated in a body.

As shown in FIGS. 1-8, body 101 is the housing for lancing device 102 and analytic device 103. The body holds and locates both the lancing device and the analytic device in proper orientation with respect to each other. The analytic device may be held in the body in a sample retrieval portion 104 that is laterally offset with respect to the lancing device. This indicates that the lancing device and the sample retrieval portion are offset angularly 141 with respect to each other about a pivot point 105 within the body. The lancing device and the analytic device are positioned in the body such that the fluid released by the lancing device is taken up into fluid inlet 106 by having body 101 direct analytic device 103 to the fluid released from the skin. More specifically, as illustrated in FIG. 20, in all embodiments of the invention the linear distance from pivot point 105 to fluid inlet 131 of analytic device 103 exceeds the linear distance from pivot point 105 to tip 102a of lancet 102. A preferred material for the body 101 is plastic. Plastic injection molding is a preferred method for fabricating the body 101. As illustrated in FIGS. 1-4, the body may be formed from one single piece. Similarly, as seen in FIGS. 5-8, the body may be formed from two pieces 107, 108 that are joined during its assembly. A preferred method for forming the body is from two separate pieces 107, 108 as shown in FIGS. 5-8. In this embodiment the two pieces 107, 108 may be joined by sonic welding, gluing, press fitting, or other means known to those skilled in the art. The body is designed to be removable and disposable, being intended for one-time use only. After one use of the sample retrieval device, the body may be discarded into a cartridge containing used devices, or into an appropriate collection device. The body incorporates several features and regions. Body 101 has a sample retrieval portion 104, tabs 109, a fluid inlet portion 106, a twist-off cap 110, a pivot point 105, an engagement portion 111, and a signal pathway shown in FIGS. 16-19.

Referring to FIGS. 1-8, sample retrieval portion 104 is formed into the body 101 during the injection molding process and is the portion of the body that receives the fluid sample. In the preferred embodiment of the present invention the body may contain an analytic device 103 that is inserted into sample retrieval portion 104. In this embodiment the sample retrieval portion of the body houses the analytic device. As seen in FIGS. 3-4, the sample retrieval portion may be defined by a slot 112 and may be equipped with tabs 109, both slot 112 and tabs 109 being sized to hold analytic device 103 in an interference fit. When the body 101 is formed from one piece, as seen in FIGS. 1-4, the slot and tabs may be formed during the injection molding process. When the body is formed from two separate pieces 107, 108, as seen in FIGS. 5-8, one of the pieces 108 may have a depression 113, illustrated in FIG. 7, formed during the molding process that creates slot 112 when the two pieces 107, 108 of body 101 are joined together. The entrance to slot 112 is defined as fluid inlet portion 106 of the body. In both of the above embodiments of the invention, as illustrated in FIGS. 4 and 6, the fluid inlet portion 106 locates fluid inlet 131 of analytic device 103, the analytic device residing in sample retrieval portion 104 of the body.

Referring to FIGS. 9-11, sample retrieval portion 104 of body 101 may be used to provide a fluid seal to analytic device 103. In a preferred embodiment of the present invention, as illustrated in FIGS. 9-11, the fluid seal is made between one surface 114 of sample retrieval portion of the body and a mating surface 115 of analytic device 103. A fluid seal is created by bringing the two surfaces 114, 115 in contact with one another, one of the surfaces having a capillary channel 116. Bringing the surfaces 114, 115 together covers the capillary channel, thereby forming a fluid capillary with the fluid capillary having the ability to direct fluid along itself. The fluid seal may be created using mechanical pressure, adhesives, or welding of plastics. Tabs 109 or a slot 112 in the sample retrieval portion of the body may provide the mechanical pressure to create the fluid seal.

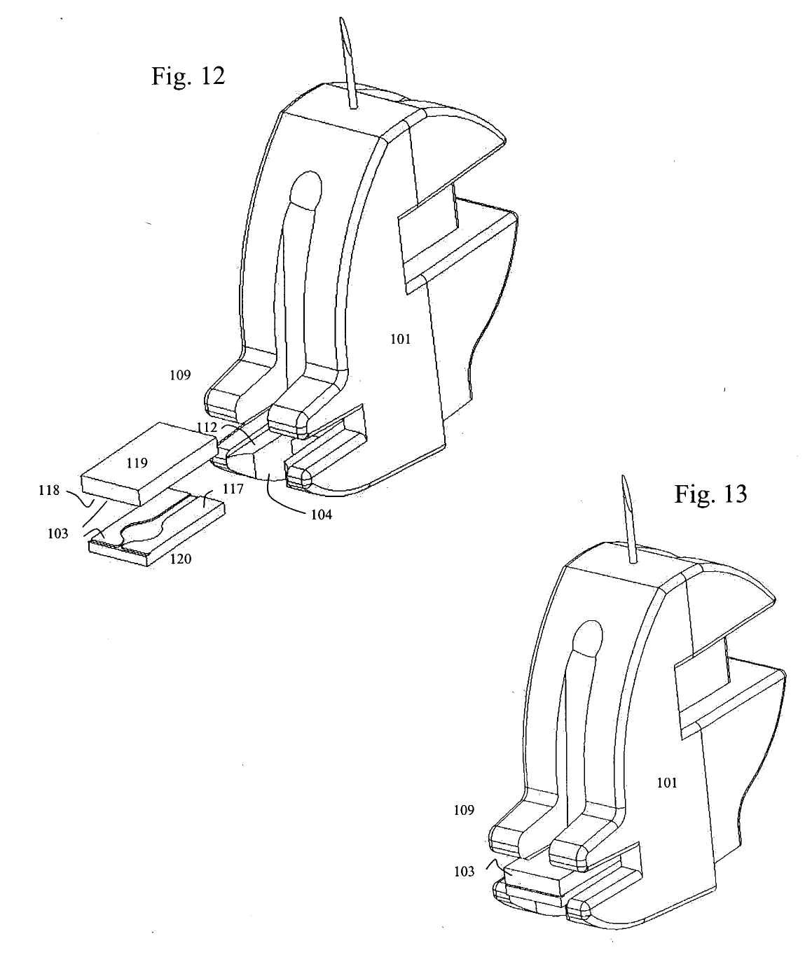

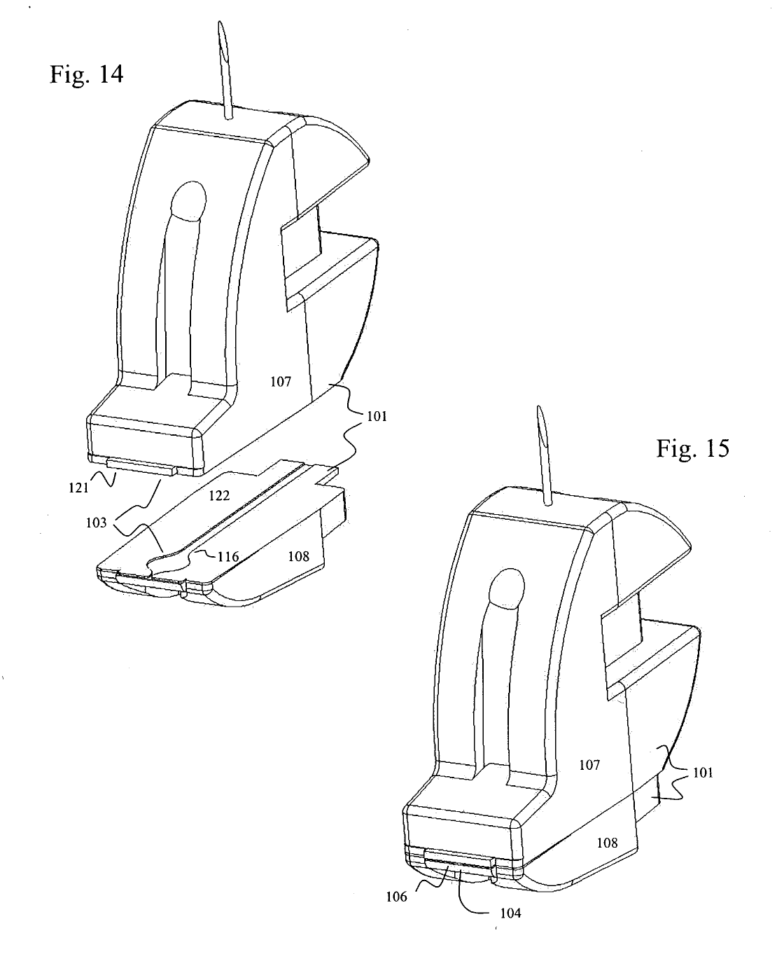

In an alternative embodiment of the present invention, as shown in FIGS. 12-13, the sample retrieval portion of the body may be used to provide a fluid seal to the analytic device by bringing two surfaces 117, 118 of the analytic device together. In this embodiment the analytic device consists of two separate members 119, 120. The fluid seal may be made between the two members 119, 120 by mechanical pressure provided by tabs 109 or a slot 112 in sample retrieval portion 104 of body 101 as described above.

In yet another alternative embodiment of the present invention, as illustrated in FIGS. 14-15, a fluid seal may be made within the body, between two surfaces of the body 121, 122 when the body is formed from two separate pieces 107, 108 as previously described. In this embodiment of the invention, a capillary channel 116 may be formed on one surface 122 of the body. The capillary channel may be formed during the injection molding process or by using a hot embossing process. In this embodiment of the present invention bringing the two halves 107, 108 of the body together creates a fluid seal as described above, designating the sample retrieval portion of the body. In this embodiment of the present invention, fluid inlet portion 106 is the area through which fluid is introduced into the sample retrieval portion, and is formed by the body. The fluid inlet preferably consists of a four-sided channel having capillary dimensions sufficient to cause fluid to flow into itself.

As illustrated in FIGS. 16-18, the body of the sample retrieval device incorporates a signal pathway 123. The signal pathway is a means of communicating the state of the fluid sample within sample retrieval portion 104 of body 101 to an analytical instrument. The signal pathway may be optical or electrical. The preferred signal pathway is electrical. In a preferred embodiment of the present invention the electrical signal pathway is along electrodes 124 in contact with and extending from a sensor 125 in the sample retrieval portion of the body to electrical contact pads 126 on the periphery of the body. The electrodes are preferably conducting traces that may be formed from any noble metal: particularly gold, platinum or silver. Conducting traces may also be formed from carbon filaments, carbon pastes, or from other materials known to those skilled in the art. In the current embodiment of the present invention, the electrodes and sensor 125, may be patterned directly onto one surface 127 of the body, when the body is formed from two pieces 107, 108 as described above. In an alternative embodiment of the present invention the sensor and the electrodes, defining the signal pathway, may be formed directly onto an analytic device, the analytic device being inserted into the sample retrieval portion of the body.

Metal electrodes may be deposited on either the body or the analytic device by either sputtering or evaporation in a vacuum chamber. Sputtering is the preferred method of deposition of metals. The metal deposited substrate is coated with a thin layer of photoresist. The photoresist is then exposed and patterned with exposure to UV light. The metal may then be etched with a reagent to create the specific metal trace patterns.

Alternatively the sensor may be constructed on a plastic substrate (polyester, polycarbonate, polystyrene) using screen-printing technology. Conductive and non-conductive inks (carbon, silver/silver chloride, gold, platinum) may be readily screen printed onto plastic substrates to form electrodes. The reagents such as glucose oxidase may then be deposited onto the electrochemical sensors. Electrochemical sensors suitable for amperometric as well as for columetric measurement may be made with screen printing.

The sensor, when used for glucose measurement, is configured based upon the fact that the enzyme glucose oxidase catalyses the oxidation of glucose to gluconic acid. The first generation glucose biosensors used molecular oxygen as the oxidizing agent. Commercially available finger stick glucose devices use a ferrocene based mediator system in lieu of molecular oxygen. Recently, immobilization techniques have been developed to “wire” an enzyme directly to an electrode, facilitating rapid electron transfer and hence high current densities.

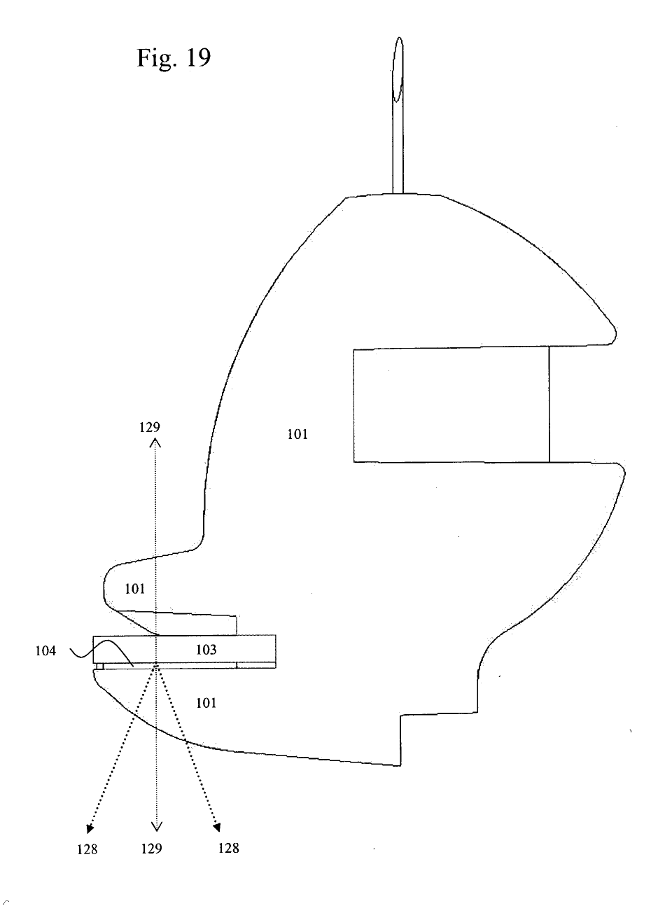

In another embodiment of the present invention, the signal pathway may be optical. Optical signal paths may be either reflective or transmissive as illustrated in FIG. 19. In this embodiment of the present invention, body 101 may be formed from an optically transparent plastic, not appreciably blocking radiation in a desired wavelength range of 600 -900 nm, allowing transmittance of an optical signal 128, 129 through the body to communicate the state of the sample in sample retrieval portion 104 to an analytic instrument. In this embodiment the chemical reaction inside the sample retrieval portion is read using either optical transmittance or optical reflectance methods. When used in an optical analysis system, either the sample retrieval portion or analytic device 103 may have reagents deposited on one surface. The reagents react with analytes in the body fluid, such as glucose or hemoglobin, to form a color change. In an optical system the signal source is a light emitting diode (LED) or a diode laser of appropriate wavelength. A preferred wavelength range is 600 nm-650 nm when the analyte is glucose. A secondary LED or diode laser may be used in the wavelength region of 700 nm-800 nm for background absorbance compensation. The optical signal is analyzed using an optical sensor present in an accompanying analytic instrument.

Referring to FIG. 19, in a reflectance system signal pathway 128 is in through one surface of body 101, to sample retrieval portion 104, through the body fluid present in the sample retrieval portion to a surface of analytic device 103, off of the surface of the analytic device and out through the same path in reverse order. In a transmittance system signal pathway 129 is in through one surface of the body, through one member of the analytic device to the sample retrieval portion, through the body fluid present in the sample retrieval portion, and out through the body in the same direction. Other variations for the optical signal pathway yielding essentially the same results are possible depending on the arrangements and variations of embodiments of the invention as discussed.

As seen in FIGS. 3-4, body 101 may incorporate a protective twist-off cap 110 covering lancing device 102. The twist-off cap may be formed as an integral part of the body during the plastic injection molding process. The twist-off cap may be sized to allow for either manual or automatic removal in order to expose the lancing device. When sized for automatic removal as shown in FIG. 3, the twist-off cap may be in the shape of a “T” in order to allow interlocking with a cartridge or carrying device. The twist-off cap serves to maintain sterility of the lancing device prior to exposure. The twist-off cap also serves as a safety device guarding from premature puncture with the lancing device.

As previously indicated, the body of the sample retrieval device may also incorporate an engagement portion 111, as illustrated in FIGS. 20-22. The engagement portion is defined by the surface of the body that couples directly to a transport instrument 130. The engagement portion allows the body to be removable. The body, through the engagement portion is attached to and removed from the transport instrument before and after a test. The engagement portion may be in the form of a slot, a hole, tabs or other common attachment means that are designed dimensionally to mate to a receptacle in the transport instrument. The engagement portion is formed into the body during the plastic injection molding process. The engagement portion may also act as a pivot point 105 in the body. The pivot point is a point within the body about which the body may rotate angularly. When pivoted about the pivot point, the body may rotate in a range from 0°-360°.

Body 101 of the sample retrieval device may vary considerably in shape and form. Illustrated in FIGS. 22A-22C, is another preferred embodiment of the present invention. In this embodiment the basic shape of body 101 is more rectilinear than the previous circular forms illustrated. The sample retrieval device as shown in FIGS. 22A-22C, contains the same features and elements described above for the previous embodiments of the invention. The sample retrieval device, as illustrated in FIGS. 22A-22C, has a body 101 that combines both a lancing device 102 and an analytic device 103 in such a way that they may be used together conveniently for lancing a skin surface and drawing a small sample of body fluid. In this embodiment body 101 is formed from 1 piece. Body 101 may be fabricated using the same techniques previously described. Body 101 has a sample retrieval portion 104, a fluid inlet portion 106, a twist-off cap 110, a pivot point 105, an engagement portion 111, and a signal pathway. All elements of this alternate embodiment of the sample retrieval device function identically as described for the previous embodiments, unless otherwise specified.

As illustrated in FIGS. 22A-22C, body 101 contains a sample retrieval portion 104. In this embodiment, sample retrieval portion 104 houses an analytic device 103 as previously described. Sample retrieval portion 104 may be formed by a slot 112 during the plastic injection molding process, slot 112 being sized to fit analytic device 103. In this embodiment analytic device 103 is preferably held in sample retrieval portion 104 by adhesives. The entrance to the slot 112 is defined as the fluid inlet portion 106 of the sample retrieval device. As previously described, fluid inlet portion 106 locates fluid inlet 131 of analytic device 103.

As illustrated in FIGS. 22A-22C, analytic device 103 be formed from either one or two separate members. When formed from one member, one surface 114 of sample retrieval portion 104 of body 101 may form a fluid seal with surface 115 of analytic device 103. The fluid seal is made by covering capillary channel 116, formed in body 101, in a manner consistent with the above descriptions for the previous embodiments.

As previously indicated the sample retrieval device, as illustrated in FIGS. 22A-22C, incorporates a signal pathway. The signal pathway may be either electrical or optical. When the signal pathway is optical the signal pathway may be the same as described above for the previous embodiments of the invention. When the signal pathway is electrical, a sensor, electrodes and contact pads may be formed either directly on surface 112 of body 101, or on one surface of analytic device 103, in a manner consistent with the techniques previously given.

As discussed the sample retrieval device contains an analytic device. The analytic device is a device for collecting and analyzing body fluid. As illustrated in FIGS. 23 and 24, analytic device 103 comprises an inlet 131, an analytic portion 132 and a vent 133, the analytic portion being in fluid communication with the inlet and vent. The analytic device may be fabricated from silicon, plastic, glass or any combination of the three. The analytic device may exist in a variety of embodiments. The analytic device may be formed from either one or two members and inserted into the sample retrieval portion of the body. As illustrated in FIGS. 9-11, in a preferred embodiment of the present invention, the analytic device is formed from one member and is inserted into sample retrieval portion 104 of body 101, whereby one surface of the sample retrieval portion of the body makes a fluid seal with the analytic device as described above. In a preferred embodiment of the present invention, the analytic device is formed from plastic using plastic injection molding or hot embossing. In an alternate embodiment of the present invention, the analytic device is formed from single crystal silicon with a [100] crystallographic orientation; however, polysilicon or other crystallographic orientations may be used. The analytic device may be formed from silicon using standard semiconductor processing techniques. These consist primarily of spin coating photoresist on a silicon wafer, exposing the photoresist to UV light with a protective mask defining the desired etching pattern, and etching the silicon, removing silicon from selected areas to form the analytic device. A preferred etching method is using a high rate plasma etcher, however, KOH (potassium hydroxide) may be used.

As seen in FIGS. 12-13, in other embodiments of the current invention the analytic device may be formed from two members, and inserted into the sample retrieval portion of the body. As previously indicated, when the analytic device is formed from two members, a sensor, reagents, and electrodes defining a signal pathway may be deposited within the analytic device in a manner consistent with techniques discussed above.

In yet another alternative embodiment of the present invention, as illustrated in FIGS. 14-15, analytic device 103 may be formed directly from body 101 without any separate members inserted into the body as described above. In this embodiment, as shown in FIG. 18, a sensor 125, reagents, and electrodes 124 defining a signal pathway 123 may be deposited within the body to form the analytic device.

Various embodiments are possible for the design of the analytic device, and are not limited to the representations described. Similarly, the signal pathway of the body may vary slightly to include various members of the analytic device and various surfaces of the body when used in alternative embodiments.

As illustrated in FIGS. 25-28, and as previously discussed, body 101 of the sample retrieval device contains a lancing device 102. The lancing device is a device for penetrating skin 134 to a depth sufficient to induce body fluid to well up to the skin surface. The lancing device is tapered at one end, coming to a point with sufficient sharpness for penetrating skin. The lancing device is preferably fabricated from metal but may also be fabricated from plastic. In a preferred embodiment the lancing device is a metal needle, protruding cylindrically from the body with one end tapered to a point. In an alternative embodiment the lancing device may be metal having the shape of a razor edge. The lancing device protrudes from the body in an orientation such that when the body is moved linearly to the surface of the skin of a user, the lancing device penetrates the skin surface. The lancing device is preferably molded into the body during the plastic injection molding process.

As illustrated in FIGS. 20-22, in all embodiments of the present invention the sample retrieval device is attached to a transport instrument 130 via engagement portion 111 of body 101. The body of the sample retrieval device is attached to the transport instrument through an engagement portion 111 located in the body and a receiving portion 135 located in the transport instrument. The receiving portion of the transport instrument may be defined as a pivot receptacle 135. The pivot receptacle engages the body at the engagement surface or pivot point 105 in such a manner that allows the body to rotate radially about the pivot point, thereby pivoting the sample retrieval device. The pivot receptacle may be cylindrical or rectangular in shape. The pivot receptacle may be sized to engage the body at the pivot point or engagement portion such that the body is held tightly in place. This may be accomplished by providing an interference fit between the body and the pivot receptacle, or by using snap fitting or interlocking features.

The transport instrument is used to move the sample retrieval device both linearly and radially within an instrument housing. Referring to FIGS. 25-28, transport instrument 130 is designed to move linearly within instrument housing 136 and in a direction parallel with linear axis 137 of lancing device 102 when the lancing device is oriented in a position for lancing. Illustrated in FIG. 25, linear axis 137 refers to a line of motion parallel with the linear shape of the lancing device and running through the center of the diameter of the lancing device. When oriented in a position for lancing, the body is positioned by the transport mechanism such that the lancing device is approximately normal to skin surface 134. In this orientation the linear axis is also approximately normal to the skin surface. When in this orientation, as depicted in FIGS. 26-27, the path followed by the body of the sample retrieval device and the lancing device is along this linear axis 137 such that the lancing device is inserted into and retracted out from the skin surface linearly at sample retrieval location 138. The sample retrieval location is the location on skin surface 134 at which the lancing takes place. This is also the location at which body fluid 139 is excised from the incision created by the lancing device.

Linear movement of transport instrument 130 may be accomplished by either mechanical or electromechanical means. When mechanical means are used, the driving force for the transport instrument may be a spring or a piston. A preferred linear driving mechanism for the transport instrument is spring loading. When a spring is used it is said that body 101 or transport instrument 130 may be “spring loaded.” Spring loaded refers to being attached to a spring or combination of springs to provide movement. Attaching the body of the sample retrieval device to a spring-loaded component provides an actuation method for lancing device 102. Spring configurations may consist of compression springs, extension springs, torsional springs or any combination of the three. Spring actuated systems may include a drive spring and a return spring to directly drive the transport mechanism. Another configuration may use a spring-loaded cam to actuate the transport mechanism and to actuate the lancing device 102. Additional components of spring-loaded systems include cocking and triggering mechanisms. Electromechanical means may also be used as the linear driving force for the transport instrument. Electromechanical means may include a motor, a solenoid, or a voice coil.

Referring to FIGS. 27-28, transport instrument 130 is also used to drive body 101 of the sample retrieval device radially within instrument housing 136. Driving the body of the sample retrieval device radially within the instrument housing is referred to as pivoting the sample retrieval device. Pivoting the sample retrieval device indicates that the body of the sample retrieval device undergoes an angular motion with respect to the instrument housing about a pivot point 105 located in the body. Pivoting the body of the sample retrieval device with respect to the instrument housing occurs after the linear retraction of lancing device 102 from sample retrieval location 138. After the lancing device has been retracted from the sample retrieval location, body fluid 139 flows out from the puncture created by the lancing device. Pivoting the body of the sample retrieval device brings fluid inlet portion 106 of the sample retrieval device into contact with fluid 139 released from skin 134 at sample retrieval location 138. As previously indicated the linear distance from pivot point 105 to fluid inlet 131 of analytic device 103 exceeds the linear distance from pivot point 105 to tip 102a of lancet 102. This placement of the fluid inlet with respect to the tip of the lancing device ensures that when the lancet has been fully retracted from the skin surface the fluid inlet will be in a position to contact the blood at the sample retrieval location. Also, requiring that the linear distance from pivot point 105 to fluid inlet 131 of analytic device 103 exceed the linear distance from pivot point 105 to tip 102a of lancet 102 ensures that the lancet tip has been completely removed from the skin surface prior to pivoting the sample retrieval device about the pivot point. If pivoting of the sample retrieval device were to occur prior to the lancing device being fully retracted from the skin surface unnecessary skin damage would occur.

A preferred mechanism for pivoting the body of the sample retrieval device with respect to instrument housing 136 is an electromechanical device. A preferred electromechanical device is a motor with a gear train that engages the transport mechanism to pivot the sample retrieval device. The transport instrument may be attached to the electromechanical device through either gears or belts or a combination of the two in order to drive the radial motion of the body.

Referring to FIG. 25, transport instrument 130 may be coupled to an analytic instrument 140 and to instrument housing 136. The transport instrument may be coupled to the analytic instrument such that the body of the sample retrieval device, when engaged to the transport instrument, is positioned such that signal pathway 128 of the body is oriented adjacent to the analytic instrument such that the state of the sample within sample retrieval portion 104 of the sample retrieval device is communicated to the analytic instrument via the signal pathway.

The analytic instrument receives and analyses a signal from analytic device 103 within sample retrieval portion 104 of body 101. The signal may be either electrical or optical as described above and is transmitted along signal pathway 128 within the body. The analytic instrument analyses the signal to determine the state of the sample within the sample retrieval portion of the body. In the case of an electrical signal the analytic instrument may monitor the change in voltage or current along conducting leads to determine the state of the sample within the sample retrieval portion of the body. In the case of an optical signal the analytic instrument 140 may incorporate of a photodiode that outputs a voltage proportional to the amount of light incident upon the photodiode. In either case an internal computational unit (ICU) interprets the signal and the results are displayed to the user. The analytic instrument is preferably located within instrument housing 136.

Instrument housing 136 is preferably a shell, preferably sized to fit in the hand, which contains the sample retrieval device 100, transport instrument 130 and analytic instrument 140. The instrument housing may also contain an actuation mechanism that interacts with and drives the transport mechanism and the sample retrieval device. The actuation mechanism may be mechanical or electromechanical. The instrument housing is designed with appropriate buttons and mechanical elements providing user interface to control actuation and analytic functions. The instrument housing may also contain a liquid crystal display (LCD) and appropriate electronics to display the results of the analysis. The instrument housing may also contain a cartridge that houses several sample retrieval devices. The instrument housing is used by placing one surface of the instrument in contact with skin surface 134 of the user and pressing the appropriate actuation buttons to initiate a test sequence.

### Methods of Using

This invention is intended to provide a disposable integrated lancing device and analytic device for use in a one-step collection and analysis of small volumes of body fluid. Body fluids that may be collected and analyzed are blood, interstitial fluid or a mixture of blood with interstitial fluid. The preferred analyte is glucose. Other analytes that may be detected include but are not limited to hemoglobin, sodium, potassium, blood gasses, and drugs of abuse. The present invention provides a device that simplifies the lancing and collection process by integrating the lancing device and analytic device into a body. The analytic device may be a nanocuvette, providing a device that is easy to fill using capillary forces. The design of the present invention is well suited for adaptation and use in either optical or electrochemical analysis systems. The invention incorporates a signal pathway into the analytic device and body for communication of analysis results. The invention is also well suited for use in a hand-held instrument housing containing an actuating, loading, and ejecting system capable of performing the necessary operations, requiring minimal manipulation from the user.

One of the most critical shortcomings of the current art is that the methods and instruments designed for body fluid sampling require two distinctly different steps: a lancing step and a filling step, which requires manual delivery of a relatively large volume of body fluid to the collection device. This two-step manual system is a very inaccurate, painful and messy method of delivering the test fluid to the collection device. Lancing devices need to be large to draw the required amount of blood. This causes a great deal of pain for the user. A good degree of dexterity is required to accurately deliver the blood to the collection device; as a result sampling is often performed improperly, requiring additional lancing.

In the present invention the lancing device and analytic device are combined within a body, and the body is located within an instrument housing. To gather a fluid sample, a surface of the instrument housing is brought into contact with the user's skin. Once contact is made between the instrument housing and the skin surface, the actuation system within the instrument housing positions the body, the lancing device, and the analytic device. The lancing device and the fluid inlet are positioned in the body so that the fluid released by the lancing device is taken up into the fluid inlet by having the body direct the lancing device and the fluid inlet to the fluid released from the skin without removing the instrument housing from the skin. This procedure facilitates an automatic lancing and sampling method for the collection of body fluid.

The automatic nature of the present invention provides a device with far greater accuracy in picking up the body fluid sample from the skin surface. As a result, producing a large volume of body fluid on the skin surface is not required for accurate collection of the sample. The greater accuracy of the automatic collection method allows for a much smaller volume of body fluid being produced on the skin surface and a much smaller lancing device than commonly used. In the present invention the lancing device is sized to cause a minimum degree of damage to the skin surface. Furthermore, fabricating a nanocuvette from silicon for use as the analytic device provides a collection device with precisely controlled volume requirements consistent with the small volumes of body fluid being produced from the lancing device. The overall system results in greater collection accuracy, lower volume requirements from the lancing device and collection device, smaller lancing device sizes, less pain and trauma for the user, and fewer if any failed tests.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The embodiments were chosen and described in order to best explain the principles of the invention and its practical application, to thereby enable others skilled in the art to best utilize the invention and various embodiments with various modifications as are suited to the particular use contemplated. It is intended that the scope of the invention be defined by the Claims appended hereto and their equivalents.

What is claimed is:

1. A device for obtaining fluid samples, the device comprising:
a body;
a lancing device at a first end of the body;
a sample retrieval portion adjacent a second end of the body and laterally offset with respect to the lancing device;
a signal pathway defined within the body and located between the first end and the sample retrieval portion; and
an engagement portion defined within the body for engaging a transport instrument.

2. A device in accordance with claim 1 where the fluid sample is blood.

3. A device in accordance with claim 1 where the fluid sample is interstitial fluid.

4. A device in accordance with claim 1 where the lancing device is a needle.

5. A device in accordance with claim 1 where the lancing device is fabricated from metal.

6. A device in accordance with claim 1 where the lancing device is fabricated from plastic.

7. A device in accordance with claim 1 where the analytic device is fabricated from plastic, glass, silicon, or any combination of the three.

8. A device in accordance with claim 1 where the body is fabricated from plastic.

9. A device in accordance with claim 1 wherein the sample retrieval portion comprises a slot for receiving an analytic device and tabs for holding the analytic device.

10. A device in accordance with claim 1 wherein the sample retrieval portion makes a fluid seal with an analytic device.

11. A device in accordance with claim 1 wherein the sample retrieval portion contains a fluid inlet.

12. A device in accordance with claim 1 where the signal pathway is a pair of electrodes.

13. A device in accordance with claim 1 where the signal pathway is optical.

14. A device in accordance with claim 1 wherein the engagement portion comprises a slot defined within the body at a side opposite the signal pathway.

15. A device in accordance with claim 1 wherein the engagement portion comprises a pivot point.

16. A device in accordance with claim 1 wherein the device is disposable.

17. A device in accordance with claim 1 further comprising a twist-off cap over the lancing device.

18. A device in accordance with claim 1 wherein the linear distance from a pivot point of the device to a fluid inlet of an analytic device in the sample retrieval portion of the device exceeds the linear distance from the pivot point of the device to the tip of the lancing device.

19. A system for analyzing fluid samples, the system comprising:
an analytical instrument;
a transport instrument coupled to the analytical instrument; and
a device for obtaining fluid samples, the device comprising:
a body;
a lancing device at a first end of the body;
a sample retrieval portion adjacent a second end of the body and laterally offset with respect to the lancing device;
a signal pathway defined within the body and located between the first end and the sample retrieval portion, the signal pathway being located adjacent the analytical instrument; and
an engagement portion defined within the body for engaging the transport instrument.

20. A system in accordance with claim 19 where the fluid sample is blood.

21. A system in accordance with claim 14 where the fluid sample is interstitial fluid.

22. A system in accordance with claim 19 where the lancing device is a needle.

23. A system in accordance with claim 19 wherein the sample retrieval portion comprises a slot for receiving an analytic device and tabs for holding the analytic device.

24. A system in accordance with claim 19 wherein the sample retrieval portion makes a fluid seal with an analytic device.

25. A system in accordance with claim 19 wherein the sample retrieval portion contains a fluid inlet.

26. A system in accordance with claim 19 where the signal pathway is a pair of electrodes.

27. A system in accordance with claim 19 where the signal pathway is optical.

28. A system in accordance with claim 19 wherein the engagement portion comprises a slot defined within the body at a side opposite the signal pathway.

29. A system in accordance with claim 19 wherein the body pivots in relation to the transport instrument

30. A system in accordance with claim 19 wherein the body is contained within an instrument housing and spring loaded within.

31. A system in accordance with claim 19 wherein the body is contained within an instrument housing driven by an electromechanical device within.

32. A system in accordance with claim 19 wherein the body is contained within an instrument housing and is both spring loaded and pivoted electromechanically within.

33. A system in accordance with claim 19 having a pivot point mated to a pivot receptacle whereby the body moves relative to the transport instrument with the pivot point and receptacle positioned opposite each other on the body and transport instrument.

34. A system in accordance with claim 19 wherein the device is disposable.

35. A system in accordance with claim 19 further comprising a twist-off cap over the lancing device.

36. A system in accordance with claim 19 wherein the linear distance from a pivot point of the device to a fluid inlet of an analytic device in the sample retrieval portion of the device exceeds the linear distance from the pivot point of the device to the tip of the lancing device.

37. A method of obtaining a fluid sample, the method comprising:
providing a sample retrieval device comprising a lancing device and sample retrieval portion;
engaging the lancing device with a subject at a sample retrieval location by moving the sample retrieval device along a linear axis defined by the lancing device;
retracting the lancing device from the subject by moving the sample retrieval device along the linear axis; and
pivoting the sample retrieval device such that sample retrieval portion is adjacent the sample retrieval location thereby allowing fluid to enter the sample retrieval portion.

38. A method in accordance with claim 37 wherein the linear distance from a pivot point of the device to a fluid inlet of an analytic device in the sample retrieval portion of the device exceeds the linear distance from the pivot point of the device to the tip of the lancing device.

39. A method of analyzing a fluid sample, the method comprising:
providing a sample retrieval device comprising a lancing device and sample retrieval portion;
engaging the lancing device with a subject at a sample retrieval location by moving the sample retrieval device along a linear axis defined by the lancing device;
retracting the lancing device from the subject by moving the sample retrieval device along the linear axis;
pivoting the sample retrieval device such that the sample retrieval portion is adjacent the sample retrieval location thereby allowing fluid to enter the sample retrieval portion;
analyzing a fluid sample at the sample retrieval location with an analytical instrument.

40. A method in accordance with claim 39 wherein the linear distance from a pivot point of the device to a fluid inlet of an analytic device in the sample retrieval portion of the device exceeds the linear distance from the pivot point of the device to the tip of the lancing device.

41. A method of collecting and analyzing fluids from skin, the method comprising:
positioning on the skin of a subject an instrument housing containing a removable, integrated lancing and analytic device for collecting fluid from the subject, the device comprising:
a body;
a lance at a first end of the body;
a sample retrieval portion adjacent a second end of the body and laterally offset with respect to the lance;
a signal pathway defined within the body and located between the first end and the sample retrieval portion; and
an engagement portion defined within the body for engaging a transport instrument within the instrument housing;
penetrating the lance into the subject by moving the lancing device along a linear axis defined by the lancing device;
retracting the lance from the subject to release fluid by moving the lancing device along the linear axis; and
pivoting the integrated lancing and analytic device so that the sample retrieval portion is adjacent the location on the subject penetrated by the lance; and
analyzing fluid that enters the sample retrieval portion through an inlet.

42. A method in accordance with claim 41 wherein the linear distance from a pivot point of the device to a fluid inlet of an analytic device in the sample retrieval portion of the device exceeds the linear distance from the pivot point of the device to the tip of the lancing device.

43. A method in accordance with claim 41 wherein the sample retrieval portion comprises a slot for receiving an analytic device and tabs for holding the analytic device.

44. A method in accordance with claim 41 wherein the sample retrieval portion makes a fluid seal with an analytic device.

45. A method in accordance with claim 41 wherein the sample retrieval portion contains a fluid inlet.

46. A method in accordance with claim 41 where the signal pathway is a pair of electrodes.

47. A method in accordance with claim 41 where the signal pathway is optical.

48. A method in accordance with claim 41 wherein the engagement portion comprises a slot defined within the body at a side opposite the signal pathway.

49. A method in accordance with claim 41 wherein the body is spring loaded within the instrument housing.

50. A method in accordance with claim 41 wherein the body is driven by an electromechanical device within the instrument housing.

51. A method in accordance with claim 41 wherein the body is both spring loaded and pivoted electromechanically within the instrument housing.

52. A method in accordance with claim 41 having a pivot point mated to a pivot receptacle whereby the body moves relative to the transport instrument with the pivot point and receptacle positioned opposite each other on the body and transport instrument.

53. A method in accordance with claim 41 wherein the fluid is analyzed optically.

54. A method in accordance with claim 41 wherein the fluid is analyzed electrochemically.

55. A method in accordance with claim 41 wherein the fluid is analyzed using fluorescence.

56. A method in accordance with claim 41 wherein the fluid is analyzed using chemiluminescence.

57. A method in accordance with claim 41 wherein the fluid is analyzed for glucose.

58. A method in accordance with claim 41 wherein the fluid is analyzed coulometrically.
